(19) United States
(12) Patent Application Publication
(10) Pub. No.: US 2002/0049450 A1
(43) Pub. Date: Apr. 25, 2002

(54) Correction of presbyopia, other refractive errors and cataract retardation

(75) Inventor: Raymond I. Myers, Collinsville, IL (US)

Correspondence Address: JOHN W KELPER, III, 7733 FORSYTH BLVD., 12TH FLOOR, ST LOUIS, MO, 63105

(73) Assignee: Second Sight Laser Technologies, Inc.

(21) Appl. No.: 09/897,585

(22) Filed: Jun. 29, 2001

Related U.S. Application Data
(63) Continuation of application No. 08/821,903, filed on Mar. 21, 1997, now abandoned.
(63) Continuation of application No. 09/312,518, filed on May 14, 1999, now abandoned.
(60) Provisional application No. 60/013,791, filed on Mar. 21, 1996.
(60) Provisional application No. 60/036,904, filed on Feb. 5, 1997.

(51) Int. Cl. A61F 9/00

(52) U.S. Cl. 606/107

(57) ABSTRACT

The invention consists of methods for treating the clear, intact crystalline lens of the eye with high energy light such as lasers, for the purpose of correcting presbyopia, other refractive errors, and the prevention of cataracts. The aim is to change the mass, shape, and/or flexure of the crystalline lens in order to maintain or reestablish the focus of all light onto the macular area.

### BACKGROUND—FIELD OF INVENTION

The invention includes methods to correct for common errors of focusing of the eye which cause the visual blurring of an otherwise precise image seen at distance or near and are now most commonly corrected with eyeglasses and contact lenses.

A major condition which cannot yet be corrected as successfully is presbyopia. Presbyopia is the functional debilitation of focusing or accommodation starting in the human between the ages of 40-50. Accommodation is the action of the ocular lens changing its shape in order to focus light precisely on the back of the retina and the image can be discerned as clear. Presbyopia is one of the few human disorders with a prevalence of 100% of the population reaching the age of the mid-50's.

Functionally, loss of accommodation is a life long reduction of focusing from birth. The focusing ability decreases throughout life from 14 Diopters at Age 10 or a capability of focusing light at 7 cm. (ff.) in front of the eye, to 8.00 D.(ff=12.5 cm.) at Age 30, 4.00 D.(ff=25 cm.) at Age 45, and 1.00 D(ff=100 cm.) at Age 52.

The anatomical structures relevant to this invention are shown in the figures starting with FIG. 1. The cornea (1) is the transparent tissue that covers the eye on the outside but allows for light to enter. The iris (2) controls the amount of light going through (i.e., pupil size), and the ocular lens (3) is just posterior to the iris. Light proceeds through the lens along the visual axis(4), strikes the back of the eye(ie., retina(5)) forms an image at the macula (6) which is transferred by the optic nerve (7) to the brain. A neural feedback mechanism from the brain causes the ocular lens to change focus by the ciliary muscle (8) according to the object in space that the individual wants to see clearly. The space between the cornea and the retina is filled with a liquid called the aqueous in the anterior chamber (9) and the vitreous (10), a gel-like substance posterior to the lens.

The lens of the eye is a multi-structural system including a central lens nucleus (12), a cortex (13) which surrounds the central nucleus (14), and a capsule (15) which envelopes the entire structure. See FIG. 2. The capsule is attached by zonules (11) to the ciliary muscle circumferentially. The cellular structure of these normally transparent tissues is ribbon-like crystallin cells resembling the structure of an onion, but with complex bundles that extend in all directions around the lens axis. Transparency is maintained by the regular architecture of crystallin through which light passes unobstructed. The older crystallin both in the cortex and nucleus have limited or no cellular functions having also lost their cell nuclei and other organelles. Aqueous does flow through the capsule to the more remote areas of the lens to provide the nutrients needed for minimal life functions and for removing toxic byproducts.

The relative shape of the lens components change throughout life and FIG. 2 shows the enlargement and changes in curvatures of the biconvex lens. The thickness of the anterior increases more than the posterior half. Additionally, thickness increases are proportionately greater in the periphery. Zonules from the ciliary muscle (11) are positioned at a different angle which has been shown to reduce the efficiency of the force that distends the lens. Until absolute (i.e., complete) presbyopia sets in, the near focusing position is when the ciliary muscle is relaxed, and the distance position is when the muscle is contracted.

It is well known that two deteriorating processes go on within the crystalline throughout life, and they become clinically obvious during the fourth decade. Presbyopia and light scattering, the first step to cataract development, occur continuously but at different rates. The possible connection between the two was clarified by a 1994 report by Koretz¹², et. al. Light scattering occurs as previously explained because individual fibers develop attachments through oxidative mechanisms, and the fibers also combine to form large, light-disrupting macromolecular complexes. Koretz studied extensively the presence of zones of light scatter. They not only confirmed that older fibers had more light scatter, but the rate of light scatter actually increased starting with the fourth decade of life. Since certain natural antioxidants within the lens are known to counteract the changes that produce light scatter, Koretz theorized that the absence of lens movement from accommodation exacerbates light scattering. Intracellular flow of fluids and natural oxidants are no longer carried through the media to keep the crystallin transparent leading to a gradual buildup of metabolic byproducts and antioxidants.

Cataracts are the opacification of the crystalline lens which are sufficient to interfere with vision and they have been extensively studied because of their high prevalence in a geriatric population. Cataracts in the aged (Sensite Cataracts) are the most common type, and are often thought as an acceleration of the previously mentioned light scatter. On the cellular level, all cataracts begin with oxidative changes of the crystallihe tissue.

Light, a portion of electromagnetic radiation, is a factor in this invention because the eye is a sensory organ that responds to “visible light”, and because photorefractive lensectomy uses high energy light. See FIG. 3. The ocular media is transparent to the visible light spectra (wavelengths of 40-700 nanometers (nm.) and the near-visible spectrum on either side have certain absorptive characteristics to different tissues. At a certain threshold ultraviolet or infrared can cause cataracts or tissue destruction. The invention utilizes controlled high energy radiation to alter the flexure of the ocular lens without causing damage to other structures.

Lasers have been used widely to correct ocular pathological conditions. This includes the repair of the hemorrhages or detachments in the retina, abnormal growth of the lens capsule after cataract surgery, and reducing intraocular pressure by creating holes in either the iris or the draining mechanism of the eye. They are often selected on the basis of which tissues are affected. For example, an excimer laser with UV light of 193 nm has been selected for photorefractive keratectomy (PRK), because it yields a focal ablation of the tissue without penetrating the cornea. Excimer lasers are available which use wavelengths from 300-350 nm. that are needed to penetrate the cornea and the lens. The area of photodisruption done by any of these lasers is exceedingly small, within the range of 0.2-0.5 microns. Infrared Nd-YTL Picosecond Lasers are used on the lens because they produce high energy photodisruption without affecting adjacent material. However, because of the higher wavelength (approx. 1056 nm.) and greater heat production, the area of photodisruption is much larger at 10-20 microns. New generation lasers for PRK are using focal, scanning, and lower powered lasers (Lin, U.S. Pat. No. 5,520,679). The focal capability makes low powered lasers possible where bundles of light below subthreshold for damage are focused to the cite of photoablation. A scanning capability prevents heat buildup by treating all regions with periods of rest.

### BACKGROUND—PRIOR ART

The traditional solution for the correction of presbyopia and other refractive errors is to provide distance glasses, reading glasses, or a combination of the two called bifocals. Other less common forms of correction include the following: 1) variable focus bifocal spectacles, 2) contact lenses, 3) aspheric photorefractive keratectomy, and 4) intraocular implant lenses for aphakic individuals. Contact fens bifocals are uncommonly used because for fitting or for technical reasons, they are optically inferior to spectacles. An additional corrective method for contact lenses called “monovision” corrects one eye for near and the other for far, and the person learns to alternate using each eye with both open. Aspheric photorefractive keratectomy (Ruiz, U.S. Pat. No. 5,533,997 and King, U.S. Pat. No. 5,395,356) provides variable focus capabilities through an aspheric reshaping of the cornea. Similar to this optical correction, some aspherical intraocular implant lenses take the place of the ocular lens in individuals following cataract surgery. All of these techniques have one or more of the following disadvantages: 1) they do not have the continuous range of focusing that natural accommodation provides; 2) they are external devices placed on the face or eye; or 3) they cut down the amount of light that normally focuses on the eye for any one particular distance.

Alternative methods to glasses have been more successful in correcting such refractive errors as myopia (nearsightedness), hyperopia (farsightedness), and astigmatism. Myopia is the most common reason for correction in a population under age 30. Astigmatism occurs generally with either myopia or hyperopia, but is occasionally the only reason for correction. Hyperopia although exceedingly common is not normally corrected until age 40's when presbyopia, or the absence of accommodation makes correction necessary.

There is a considerable interest in the ophthalmic field to explore various methods of refractive surgery for correction of presbyopia and other refractive errors. TR Werblin (Pat. No. 5,222,981)³ proposed the surgical removal of the clear, intact crystalline lens for the purpose of correcting presbyopia and other ametropias, and substituting a multiple interchangeable components-intraocular lens. This initiated an editorial in 1992 by George Waring entitled, “Presbyopia and accommodative intraocular lenses—the next frontier in refractive surgery?”⁴ and he recognized the feasibility and attractiveness of resolving a refractive error that afflicts 100% of individuals by their 50th decade.

Using a laser to somehow change the lens to correct for presbyopia and hyperopia, as listed herein was proposed in patents by Ronald A Schachar⁵ first in Nov. 1995. In his Pat. Nos. 5,529,076, 5,503,165, 5,489,299, and 5,465,737, he proposes a series of claims predominantly based upon a preferred method of changing the outside of the eye or sclera to restore accommodation. In addition, he suggests a alternative embodiment involving the use of radiation on the lens. As will be described later, this is perhaps the first reference of treating the lens for the purpose of correcting presbyopia, but Schachar's method treats entirely different structures and works according to a different mechanism than the invention described herein.

The commercial possibilities for accepting the methods of my invention include the following observations:

This is expected to be a relatively simple outpatient procedure once it is developed with no further inconvenience than photorefractive keratectomy (PRK) of today;

100% of the population over the age of 50 are affected by presbyopia, a percentage that is at least double the number of individuals who wear corrective eye wear for other reasons such as myopia, hyperopia, and astigmatism;

Because the procedures used in correcting presbyopia have a possible therapeutic effect in slowing cataract formation, there are possible secondary health preventative benefits; and

PRK might be used in conjunction with PRL, perhaps during the same surgery and with the same instrument to eliminate presbyopia as well as other refractive errors.

The surgical alternatives include the possibility of correcting myopia, astigmatism, and hyperopia and therefore accomplishing all refractive surgery inside the eye without the risks of external surgical exposure Other types of possible correction for presbyopia have been proposed including surgery on the sclera, also called a sclerectomy; and the removal of the clear, intact crystalline lens leaving the lens capsule where a silicone injection would return a flexible lens, assuming the continued functioning of the ciliary muscle.

### OBJECTS AND ADVANTAGES

The feasibility of photorefractive lensectomy is based upon a unique concept and an extensive review of the literature which describes ocular properties that are favorable to correct presbyopia and other conditions described herein.

The originality begins with the fundamental idea of the surgery: that is, directed high energy light can reduce the volume, mass, and increase flexibility by treating the clear, intact crystalline lens for the purpose of correcting presbyopia, other refractive errors, and for cataract prevention. The use of a laser in order to remove cataracts (L'Esperance, U.S. Pat. No. 4,538,608, Krasnov, U.S. Pat. No. 3,971,382) including discreet, focal cataracts,⁶ has been proposed. Exogenous factors such as nutritional supplements to enhance accommodation have been considered along with its possible reduction in cataract development. In addition, behavioral optometrists proposed many years ago the use of focusing exercises to slow down the deterioration of lens accommodation. None of these has been widely accepted.

There are possible benefits that come from altering the flexibility of the ocular lens and reestablishing circulation of the nutrients to tissue formed many years before. Reestablishing aqueous circulation to areas having age-related changes can reduce later changes that affect presbyopia and cataract development. PRL (photorefractive keratectomy) maintains the continuous focusing capability in all positions of gaze without external optical and external means.

This invention presents a strategy that alters old lens fibrils which do not possess significant metabolic function including germinal development. In contrast to the alternative embodiments in his patents, Schachar proposes to treat the epithelium controlling the germinal growth of the lens, which may be subject to toxic or mutagenic changes.

It should be noted that the surgical techniques included within this invention are less invasive surgery than techniques of presbyopic correction that are now being proposed by others. That is, they all require surgical incision, whereas the techniques of the invention require radiation penetration, but not surgical incision. Even current refractive surgical techniques require surgical incision and exposure to the outside

The crystalline lens enclosed in its fibrous capsule represents an independent system segregated from rest of the eye. Nothing that happens inside the intact sac will later affect the rest of the eye which suggests a good degree of independence from the rest of the eye. The short term considerations of internal lens alteration are significant in heath and safety considerations, but the long term effects should not be so much of a concern.

Understanding different types of refractive error help to explain why surgical changes with this invention have more flexibility than the current FDA-approved photorefractive keratectomy (PRK). In PRK the laser produces a unique and calculated shape to the corneal surface that must precisely focus light at the retina Because of significant focusing capability, the PRL patient sees at both distance and near, when slightly over corrected or hyperopic. This is a typical occurrence we see in hyperopes without PRL who do not require correction until they reach early presbyopia where the absence of accommodation allows only one focal distance.

Further objects and advantages of the invention will become apparent from a consideration of the drawings and ensuing description.

### DRAWING FIGURES

FIG. 1⁷ shows the pertinent gross anatomy of the eye related to the invention.

FIG. 2⁸ is an enlargement of the crystalline lens with increasing age and the different effects upon the shapes and hardness.

FIG. 3⁹ shows the range of light in the near and visible spectrum to which the eye is normally exposed or which is used for photorefractive lensectomy.

FIG. 4 Shows the major embodiments necessary for the instrumentation of the invention.

FIG. 5 summaries the different strategies that would carry out the major embodiments of the invention.

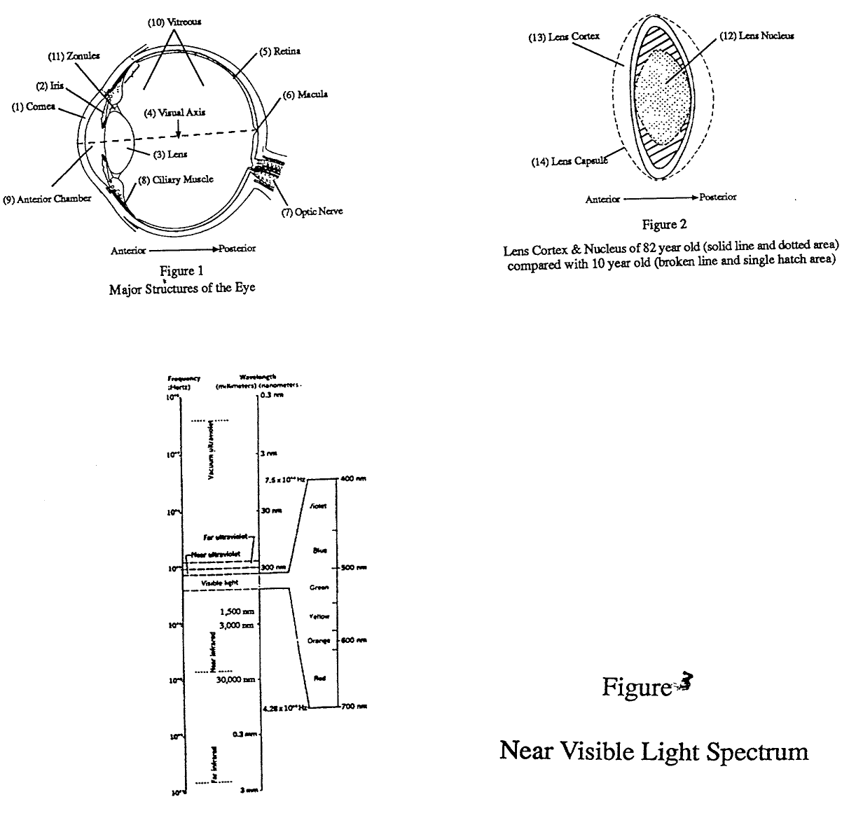

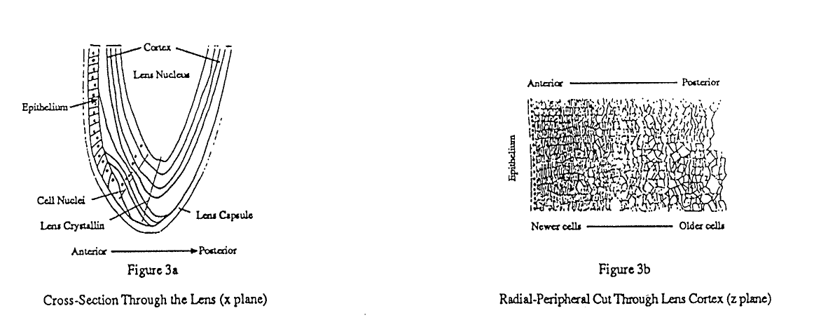

### SUMMARY OF INVENTION

The invention recognizes that the intact crystalline lens can be treated safely with a focused, scanning laser to restore or increase accommodation by reducing the volume, particularly of the lens cortex, and/or by softening the lens, particularly the nucleus. This would be carried out by the ablation or removal of crystallin within the older, more inert areas of the crystalline lens through a series of cavities, microspheres, and/or microchannels, possibly with concomitant antioxidative therapy to minimize acute radiation exposure. An additional benefit is to increase the fluid transport system of older tissue by maintaining radical scavenger systems that affects light scatter and cataractogenesis. These procedures are called “photorefractive lensectomy” or “crystalline lens modulation.” In addition, the methods are used to correct for the refractive errors of myopia, hyperopia, and astigmatism.

### DESCRIPTION

The invention consists of methods for treating the clear, intact crystalline lens of the eye with high energy light such as from lasers, for the purpose of correcting presbyopia, other refractive errors, and the prevention of cataracts. The aim is to change the mass, shape, and/or flexure of the crystalline lens in order to increase the focusing capacity of the eye.

Photorefractive lensectomy would be performed as ophthalmic surgery in an outpatient setting without general anesthesia. The patient is prepared as in cataract surgery or laser surgery but without the necessity of intraocular incision. The anterior segment of the eye is prepared by procedures that are common to regular vision testing including topical anesthetic, dilating drops, and cycloplegia (temporary paralysis of the accommodation). An A-Scan or similar instrument measures the exact dimensions of the lens including the geometric center, thickness and other contour measurements of the nucleus and cortex. After the eye is stabilized in position with fixation forceps, the patient is then situated under the instrument and the eye is aligned with the surgeon viewing through a binocular microscope (28). See FIG. 4. The microscope allows the surgeon to visualize the lens structure, and locate the starting point as well as to observe the treatment. Two separated Helium-Neon non-therapeutic laser beams (22) used for alignment are seen through the binocular microscope and the focused images coincide at the cite of focus. A foot pedal enables the surgeon to control the progress of the ablation.

High energy light is formed by a laser with light that passes through the cornea and the aqueous, and is then focused at a precise location (23) in the ocular lens. See FIG. 4. A collimated beam (20) is produced by the laser (21) which is split by internal optics and/or apertures (22) to produce multiple light bundles. They are at subthreshold energy levels when they travel through the cornea and the lens, except at the point of focus where the photoablation and/or photodisruption occur. The high energy beam is capable of traversing the X(25), Y(26), and Z(27) axes representing three dimensional movement from the original starting point. They are combined with the lens biometric measurements, visual inspection, the treatment strategy and algorithms into a computerized program which drives the procedure under the surgeon's control. The computer controls the laser location, energy level, and the number of pulses and duration. A scanning program enables the beam to alternate between different locations in order to minimize local heat build up. The angle (24) of the incoming focused light to the ablation site will be calculated and adjusted by a computer to 1) utilize the lens nucleus as a masking background; 2) ensure that light will not end in the perimacular region; and 3) avoid exposure to the ciliary body.

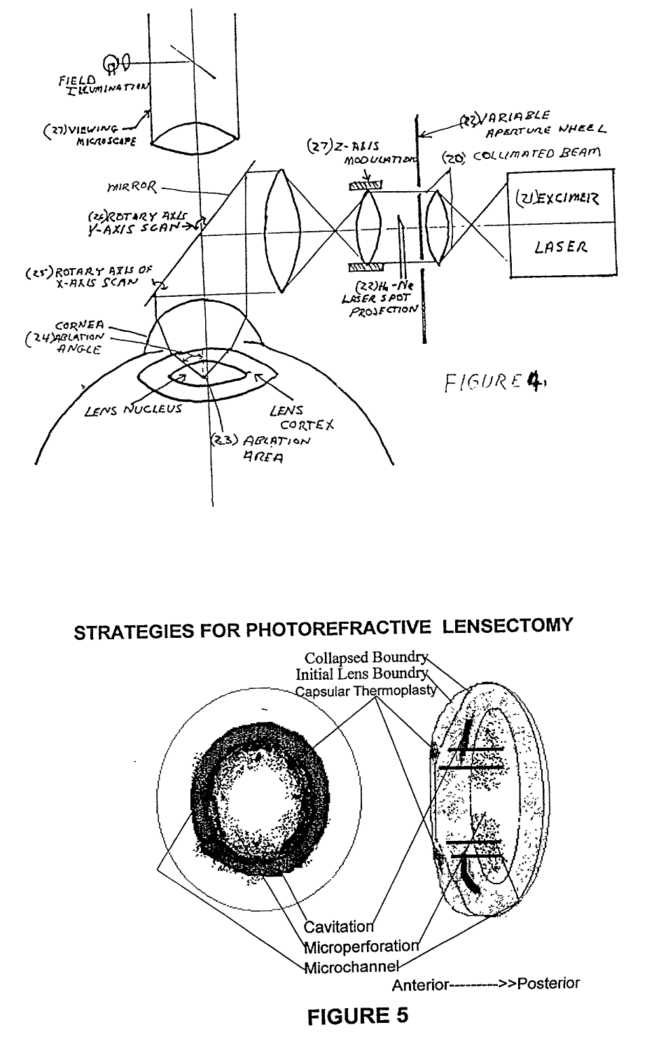

At least four strategies for PRL (photorefractive lensectomy) are possible all of which succeed in altering the internal lens structure, or preparing the lens system for greater movement or fluid exchange. Modifications are more likely in the periphery than within the visual pathways. A previously cited article⁶ has found that the Nd-YTL Pico-Second Laser can safely remove an isolated cataract in an otherwise transparent lens without types of radiation when used in the crystalline lens produce ablation without any expansion of the ablated area occurs permanently. Strategies for performing photorefractive lensectomy are demonstrated in FIG. 5 and are as follows:

1) volume reduction through cavitation. That is, joining the individual small ablations to produce a larger cavity void of lens fibers outside the visual pathway that reduces peripheral volume;

2) softening or increasing flexibility by multiple small ablations applied sparsely to large areas of the nuclear region;

3) reduction of lens capsule outside the germinal epithelial region of the epithelium through thermoplasty to form a firmer lens sac after volume reduction and.

4) produce microchannels or a linear pattern of connected microspheres traversing parallel to the visual axis (4) between the older tissue in the depths of the lens to the newer tissue in the anterior cortex, for the purpose of enhancing fluid flow between older and newer crystallin.

Selective volume reduction is the preferred strategy, because of the predominance based upon current thinking of peripheral volume increases as the cause of presbyopia. The cavities can also follow the architecture of the lens to reduce the numbers of fibers that are interrupted. Such cavities when produced within the lens structure cause the soft lens capsule to collapse providing a thinner equatorial width and a different angular insertion to the zonules, and a more efficient ciliary muscle action. See FIG. 5.

The presentation of microspheres to the harder nucleus is a softening technique applied primarily to the peripheral nucleus to avoid the visual axis. Because of the likelihood of multiple causes of presbyopia, including the role of a hardening nucleus with age change.

If mass is removed from the lens cortex, the lenticular capsule may well loosen and reduce the useful energy imparted on the zonules. If necessary the capsule which has significant elastic tissue can be tightened by thermoplasty using infrared radiation without opening the capsule.

The placement of microchannels for greater flow of fluids throughout the lens supplements the major fluid flow that occurs by the “squeezing.” See FIG. 5. They are multiple spheres that are connected and parallel the anterior-posterior path of light Presently, the preferred group to carry out this treatment is myopic subjects with spectacle prescriptions of less than 3.00 D. Patients would be pre-presbyopic in their early 40's, with 3-5 Diopters of accommodation, and have undergone a full-dilated eye exam to determine the following:

1) No prior history of eye disease, trauma, cataracts, or glaucoma.

2) No family history of early cataract development, glaucoma, or collagen vascular disease.

3) Normal gonioscopic findings.

4) No significant personal systemic diseases.

High energy light from 100-1,500 nm can be produced by various types of laser light sources. Depending upon the characteristics of the light and the treatment strategy, the treatment occurs through the processes of photoablation, photofragmentation, photoemulsification, photoacoustics, and/or thermal decomposition. The preferred type of radiation is ultraviolet light in the range of 310-350 nm. and infrared light (800-1,500 nm). See FIG. 4. It is difficult to predict which end of the spectrum is the more useful, because of advantages and possible pitfalls of each. The advantages of the UV light are that ablation is more self-contained within the treated area, with a less likelihood of induced opacification. This is likely to be more useful with the microspherule method. UV light is absorbed by the cornea up to about 300 nm, and over this a relatively small amount (less than 1%) is absorbed which is not likely to affect the cornea at lower energy levels and at very high pulse rates. Infrared light has the least potential to damage the cornea or any other tissue before being focused. Its larger ablation area may not matter if tissue is removed peripherally as in the cavitation method where some fiber disruption does not interfere with vision. Both have been done already in the cornea where a dosed cavity was formed in the fibrous, corneal tissue (intrastromal keratectomy). Tissue in very close proximity was not affected because of the concentrated action at the focal point of photodestruction, and the relative inertness of vaporous byproducts.

Steps taken to maximize the safety and efficacy to the lens and other vital parts of the eye include the following:

treat the older and inert areas of the lens cortex and nucleus;

control the angle of the laser beam such that extraneous light incident on the ablation area is masked by inert posterior tissues;

minimize pathological changes to the cornea, equatorial (germinal) lens epithelium and crystallin, ciliary body, and the perimacular region of the retina.

reduce debris and ionic free radicals which enter the aqueous and vitreous post surgically; and

keep from physically destroying the lens capsule thereby allowing the lens contents to have an opening to the aqueous.

### DESCRIPTION—ALTERNATE EMBODIMENTS

The alternative embodiments of this invention are the correction of myopia, hyperopia, and astigmatism, predominantly because they can be performed independent to the correction of presbyopia. They are also already corrected by existing refractive surgery techniques. Myopia is the ocular condition where light from a distant object focuses in front of the retina. In hyperopia, a distant image focuses in back of the retina. In either cased changing the lenticular curvature and/or the refractive strength (ie., refractive index) alter the focus. Astigmatism is a refractive error defect where the focus in one direction is different than the focus at 90 degrees from it, thus distorting an image like a basketball into an ellipse.

When correcting different ametropias, the cavity location in FIG. 5 will vary as illustrated. Cavities toward the optical center cause a surface collapse that produce a less convex anterior surface and will reduce myopia. Alternatively, placing the cavity toward the equator reduces hyperopia. Creating a cavity of varying and regular thickness change induces lenticular astigmatism which counteracts the existing astigmatism.

The connection between presbyopia and the development of light scatter and lens opacification makes cataract prevention an alternate embodiment and independent of the success of presbyopia.

An alternate embodiment is a more invasive form of surgery which relates to this invention by representing an alteration of the dear crystalline lens for the purpose of the previously mentioned indications. The aim of the surgery is to create a large cavity centrally; to liquefy and aspirate the contents through a small opening and tube which in itself does not interfere with vision; and to replace the former contents with an artificial and inert liquid. This might be done to produce large changes of refractive error occurring in a small number of individuals.

An alternate embodiment would be the use of a probe for the delivery of laser energy inserted through a corneal incision which has already been suggested for the treatment of a cataractous lens.¹⁰ The probe abuts the lenticular surface and would be used when and if high energy does not penetrate the cornea or is not transported through the aqueous.

An alternate embodiment is the use of concomitant drugs to reduce inflammation and the effects of free radicals and debris within the lens or other tissues before and after the procedure. Antioxidative drugs such as galactose, glutathione, and penicillin penetrate the lens matrix through the established and newly created channels, and can react locally with any active by-products of the treatment. Also, mydriatics and cycloplegics are used at the time of the treatment. Miosis for pressure control, cortico-steroids and/or non-steroidal anti-inflammatory drugs (NSAIDS) are also used after surgery. An antioxidant such as reduced glutathione and penicillamine would likely be prescribed to the patient before and after surgery to facilitate the reduction of free radicals created during the surgery.

### CONCLUSIONS, RAMIFICATIONS, AND SCOPE

The properties of the crystalline lens and lasers identified herein suggest the feasibility of treating the clear, intact, crystalline lens for the purpose of correcting presbyopia, refractive errors, and other disease conditions including cataract prevention. A multiplicity of techniques makes it possible to address the different and probable causes of presbyopia. The result of the treatments is to postpone presbyopic development for 10-15 years and restore from five to eight diopters of accommodation. All of these changes will be under the control of a computerized laser, where modifications can combine presbyopic as well as myopic, hyperopic or astigmatic changes.

Considering alternative means of correcting presbyopia in refractive surgery, these methods in this invention are in general less invasive and traumatic to current proposed methods.

The following represents important information that suggests that photorefractive lensectomy is feasible:

the ciliary muscle can control the restored accommodation because it retains and increases its strength at least until the age of 60;

certain sections of the ocular lens become a nuclear with little or no metabolic activity;

differences in the composition of the deeper areas of especially the cortex suggest not only an imperviousness to the effects of radiation but an absence of defense mechanisms to compensate for lasing;

the physiology of cataract formation is well known and appears to be an exaggerated response to the same physiological changes in light scatter, which appears to be related to presbyopia; and

there is a favorable masking effect proceeding from an anterior to posterior through the eye. The cornea will transmit the effective ranges which are then masked by the lens nucleus.

Photorefractive lensectomy is an extension of the current photorefractive keratectomy. It is also clear that the age group who is currently interested in PRK (ie., 430-40 year old individuals) may benefit most from PRL. The feasibility of using them together may well depend upon an instrument that is capable of operating on the cornea, and being adaptable enough to meet the needs of lens ablation or photodisruption. Although the first FDA approved lasers are with UV light, experimental work is also being done with infrared light using short-pulsed beams. Given the possible adaptability of PRL to specific UV or even infrared radiation, there are opportunities to head in either direction. Tunable lasers exist that provide different wavelengths.

It is also possible that photorefractive lensectomy could become a new generation of refractive surgery, capable of resolving all common indications including those now done on the cornea.

I claim

1. A method for the correction of refractive errors of the eye and other ocular anomalies.
